(19) United States
(12) Patent Application Publication
(10) Pub. No.: US 2025/0281435 A1
(43) Pub. Date: Sep. 11, 2025

(54) CHEWABLE GUMMY CONTAINING BETA-HYDROXY BETA-METHYLBUTYRIC ACID (HMB)

(71) Applicant: Concept Matrix Solutions, Newbury Park, CA (US)

(72) Inventors: Sevan Kesenci, Woodland Hills, CA (US); Robert Davidson, Woodland Hills, CA (US); David Reid, Woodland Hills, CA (US); Naji Abumrad, Woodland Hills, CA (US); Tony LaRosa, Woodland Hills, CA (US)

(21) Appl. No.: 19/072,429

(22) Filed: Mar. 6, 2025

Related U.S. Application Data
(60) Provisional application No. 63/561,931, filed on Mar. 6, 2024.

(51) Int. Cl. A61K 31/19; A23G 3/36; A23G 3/42; A23G 3/44; A23G 3/48; A61K 9/00; A61K 47/12; A61K 47/36; A61K 47/42

(52) U.S. Cl. CPC A61K 31/19; A23G 3/368; A23G 3/42; A23G 3/44; A23G 3/48; A61K 9/0056; A61K 47/12; A61K 47/36; A61K 47/42

(57) ABSTRACT

The present invention provides for a chewable gummy containing β-hydroxy β-methylbutyric acid (HMB), a method of making and using the same, as well as the slurry used to form the chewable gummy.

### CROSS REFERENCE TO RELATED APPLICATIONS

The present application claims the benefit of priority to U.S. Provisional Application No. 63/561,931, filed Mar. 6, 2024, the contents of which is hereby incorporated by reference.

### BACKGROUND OF THE INVENTION

Gummies, also known as gummi candies, gummy candies, or jelly sweets, are a popular category of chewy confections that have gained widespread appeal, particularly among children. These gelatin-based treats come in various shapes, sizes, and flavors, with iconic forms including gummy bears, Sour Patch Kids, Jelly Babies, and gummy worms. Numerous brands, such as Bassett's, Haribo, Albanese, Betty Crocker, Hersheys, Disney, and Kellogg's, produce an array of gummy snacks, often targeting young consumers with colorful and playful designs. US 2019 0364924 A1 (“Method and composition for the preparation of gelatin based gummy candies”); TGMACHINE, Gummy Manufacturing Process: Complete Guide (https://tgmachine.com/gummy-manufacturing-process/) (accessed Feb. 18, 2025).

The primary ingredients in gummy candies typically consist of corn syrup, sucrose, gelatin, starch, and water. To enhance their sensory appeal, manufacturers incorporate small quantities of coloring and flavoring agents. Food acids like citric acid and malic acid are frequently added to impart a sharp, tangy flavor to the gummies. In recent years, to cater to vegan and vegetarian consumers, alternative gelling agents such as starch and pectin have been utilized in place of gelatin, expanding the market reach of these popular confections. US 2019 0364924 A1 (“Method and composition for the preparation of gelatin based gummy candies”); TGMACHINE, Gummy Manufacturing Process: Complete Guide (https://tgmachine.com/gummy-manufacturing-process/) (accessed Feb. 18, 2025); US 2018 0103655 A1 (“Hard Candy with Gummy Center and Systems and Methods for Making Same”).

While gummies have proven to be a successful platform for delivering vitamins and supplements, their use in nutraceutical applications faces several challenges and concerns. One significant obstacle is the limited amount of active ingredients that can be incorporated into a single serving, often falling short of the levels found in traditional pill forms. This limitation may necessitate consumers to ingest multiple gummies to achieve the recommended dosage. Thomas, P., “Gummies Functional gummies: the (still) rising star of the nutraceutical space . . . and the production challenges brands can face on the ascent” (https://digital.teknoscienze.com/agrofood_industry_hi_tech_34_2_2023/gummies_-_functional_gummies_the_still_rising_star_of_the_nutraceutical_space) (accessed Feb. 18, 2025). Additionally, the high sugar content typically used to mask unpleasant tastes or odors of certain nutraceuticals can be problematic from a health perspective. Streit, L., “Do Gummy Vitamins Work? The Benefits and Downsides,” Oct. 20, 2021, Healthline (https://www.healthline.com/nutrition/gummy-vitamins) (accessed Feb. 18, 2025). The production process itself poses challenges, as the heat involved in traditional manufacturing methods can degrade thermosensitive ingredients like Vitamin C, potentially compromising their efficacy. Chu, W., “Melding gummy appeal with nutrient dosage delivery: Challenges and solutions,” (https://www.nutraingredients.com/Article/2021/12/16/Melding-gummy-appeal-with-nutrient-dosage-delivery-Challenges-solutions/) (accessed Feb. 18, 2025). Cross-contamination risks associated with the conventional starch-molding method have also raised concerns about maintaining hygienic production conditions for nutraceutical gummies. Thomas, P., “Gummies Functional gummies: the (still) rising star of the nutraceutical space . . . and the production challenges brands can face on the ascent” (https://digital.teknoscienze.com/agrofood_industry_hi_tech_34_2_2023/gummies_-_functional_gummies_the_still_rising_star_of_the_nutraceutical_space) (accessed Feb. 18, 2025); Chu, W., “Melding gummy appeal with nutrient dosage delivery: Challenges and solutions,” (https://www.nutraingredients.com/Article/2021/12/16/Melding-gummy-appeal-with-nutrient-dosage-delivery-Challenges-solutions/) (accessed Feb. 18, 2025). Furthermore, the stability and potency of vitamins in gummy formulations can be inconsistent over time, leading to potential discrepancies between the labeled and actual nutrient content. Rodriguez, T., “Dangers of Gummy, Patch, and Powder Vitamin Supplements,” Nov. 11, 2022, Med Shadow Foundation (https://medshadow.org/gummy-vitamins/) (accessed Feb. 18, 2025). Lastly, the appealing candy-like appearance of gummy supplements increases the risk of overconsumption, particularly among children.

β-Hydroxy β-methylbutyric acid (HMB) is a naturally occurring metabolite of leucine that plays a role in protein metabolism and has been shown to have potential benefits for muscle growth, strength, and recovery. It is used as a dietary supplement and in medical foods to support muscle health, particularly in conditions associated with muscle wasting.

Orally delivering HMB presents several challenges that need to be addressed for effective supplementation. One of the primary obstacles is its unpleasant taste and odor, which can make it difficult for individuals to consume consistently, potentially leading to poor adherence to supplementation regimens. The relatively large dose required for efficacy, typically 3 grams per day, further compounds this issue, as it may necessitate multiple administrations throughout the day. Wikipedia, “β-Hydroxy β-methylbutyric acid” (https://en.wikipedia.org/wiki/% CE %92-Hydroxy_% CE % B2-methylbutyric_acid) (accessed Feb. 18, 2025); Raman, R., Healthline, Nov. 27, 2024, “Benefits, Downsides, and Dosage of Hydroxymethylbutyrate (HMB),” (https://www.healthline.com/nutrition/hmb) (accessed Feb. 18, 2025). Additionally, the absorption and bioavailability of HMB can be affected by various factors, including the presence of food and the timing of ingestion relative to exercise. Wilson G J, Wilson J M, Manninen A H. Effects of beta-hydroxy-beta-methylbutyrate (HMB) on exercise performance and body composition across varying levels of age, sex, and training experience: A review. Nutr Metab (Lond). 2008 Jan. 3; 5:1. doi: 10.1186/1743-7075-5-1. PMID: 18173841; PMCID: PMC2245953. The peak plasma concentration of HMB occurs approximately 1-2 hours after ingestion, which may require careful timing of supplementation to maximize its effects. Id.

### SUMMARY OF THE INVENTION

The present invention provides for a chewable gummy containing β-hydroxy β-methylbutyric acid (HMB), a method of making and using the same, as well as the slurry used to form the chewable gummy.

In one embodiment, the invention provides for a chewable gummy containing water, gelatin, sugar, pectin, tapioca syrup, raspberry concentrate, citric acid, malic acid, potassium sorbate, sodium citrate, Magnasweet MM100, HMB, acid flavor reducer, and flavoring agent.

In one embodiment, the invention provides for a chewable gummy formed from water, gelatin, sugar, pectin, tapioca syrup, raspberry concentrate, citric acid, malic acid, potassium sorbate, sodium citrate, Magnasweet MM100, HMB, acid flavor reducer, and flavoring agent.

In another embodiment, the invention provides for a chewable gummy containing water, gelatin, sugar, pectin, tapioca syrup, raspberry concentrate, citric acid, malic acid, potassium sorbate, sodium citrate, Magnasweet MM100, HMB, acid flavor reducer, and one or more of mountain berry flavor, mixed berry flavor, watermelon flavor, pineapple flavor, and green apple.

In another embodiment, the invention provides for a chewable gummy formed from water, gelatin, sugar, pectin, tapioca syrup, raspberry concentrate, citric acid, malic acid, potassium sorbate, sodium citrate, Magnasweet MM100, HMB, acid flavor reducer, and one or more of mountain berry flavor, mixed berry flavor, watermelon flavor, pineapple flavor, and green apple.

In one embodiment, the invention provides for a slurry containing water, gelatin, sugar, pectin, tapioca syrup, raspberry concentrate, citric acid, malic acid, potassium sorbate, sodium citrate, Magnasweet MM100, HMB, acid flavor reducer, and flavoring agent.

In one embodiment, the invention provides for a slurry formed from water, gelatin, sugar, pectin, tapioca syrup, raspberry concentrate, citric acid, malic acid, potassium sorbate, sodium citrate, Magnasweet MM100, HMB, acid flavor reducer, and flavoring agent.

In another embodiment, the invention provides for a slurry containing water, gelatin, sugar, pectin, tapioca syrup, raspberry concentrate, citric acid, malic acid, potassium sorbate, sodium citrate, Magnasweet MM100, HMB, acid flavor reducer, and one or more of mountain berry flavor, mixed berry flavor, watermelon flavor, pineapple flavor, and green apple.

In another embodiment, the invention provides for a slurry formed from water, gelatin, sugar, pectin, tapioca syrup, raspberry concentrate, citric acid, malic acid, potassium sorbate, sodium citrate, Magnasweet MM100, HMB, acid flavor reducer, and one or more of mountain berry flavor, mixed berry flavor, watermelon flavor, pineapple flavor, and green apple.

In another embodiment, the invention provides for a method that includes orally administering to a human the chewable gummy described herein.

### BRIEF DESCRIPTION OF THE DRAWINGS

FIG. 1 shows a Method of Manufacturing Chewable Gummy Containing β-Hydroxy β-Methylbutyric Acid (HMB) according to Example 2.

### DETAILED DESCRIPTION OF THE INVENTION

The present invention provides for a chewable gummy containing β-hydroxy β-methylbutyric acid (HMB), a method of making and using the same, as well as the slurry used to form the chewable gummy.

The present invention can be more readily understood by reading the following detailed description of the invention and study of the included examples.

As used herein, the following terms have the meanings ascribed to them unless specified otherwise.

The terms “comprise,” “comprising,” “include,” “including,” “includes,” “contains,” and “containing” when used in this specification and claims are intended to specify the presence of stated substances, features, integers, components, or steps, but they do not preclude the presence or addition of one or more other substances, features, integers, components, steps, or combinations thereof.

The term “about” modifies the subject values, such that they are within an acceptable error range, as determined by one of ordinary skill in the art, which will depend in part on the limitations of the measurement system.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “Acid Flavor Reducer” refers to a food additive specifically formulated to decrease the perception of sourness or acidity in the chewable gummy containing β-hydroxy β-methylbutyric acid (HMB) without significantly altering its pH. This ingredient helps balance flavors, reduce tartness, and/or mask the potentially unpleasant taste of HMB, thereby improving the overall palatability and consumer acceptance of the gummy supplement.

The term “β-Hydroxy β-Methylbutyric Acid” or “HMB” (otherwise known as its conjugate base, “β-hydroxy β-methylbutyrate”), is a naturally produced substance in humans that is used as a dietary supplement and as an ingredient in certain medical foods. HMB is present in insignificant quantities in certain foods, such as alfalfa, asparagus, avocados, cauliflower, grapefruit, and catfish. HMB has the IUPAC name 3-hydroxy-3-methylbutanoic acid, formula C₅H₁₀O₃; CAS No. 625-08-1, INII 3F752311CD, molar mass 118.132 g·mol⁻¹, and structural formula shown below.

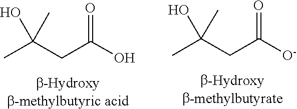

The term “chewable gummy” refers to a semi-solid, elastic oral dosage form designed for the delivery of active ingredients, such as hydroxymethylbutyrate (HMB). This dosage form is intended to be chewed before swallowing, providing a convenient and palatable method of administration. Chewable gummies maintain their molded shape and yield to mastication, offering a texture that is both appealing and functional for oral consumption. The composition of a chewable gummy typically includes one or more gelling agents, which provide the characteristic chewy texture and structure. These gelling agents may be combined with other ingredients such as sugars, water, sweeteners, and flavoring agents. The sweeteners and flavoring components serve to enhance patient acceptance and potentially mask any undesirable tastes associated with the active ingredients. In the context of delivering HMB, the chewable gummy format offers several advantages. It allows for the incorporation of a precise dose of HMB within a matrix that can be easily consumed and may improve compliance due to its appealing form. The gummy composition can be formulated to include additional beneficial ingredients, such as vitamins or probiotics, to create a comprehensive nutritional supplement. The chewable gummy dosage form represents an innovative approach to pharmaceutical and nutraceutical delivery, combining the benefits of accurate dosing with improved palatability and ease of administration. This format is particularly suitable for delivering substances like HMB, which may benefit from a more consumer-friendly presentation compared to traditional pill or powder formats.

The term “Citric Acid” refers to an organic acid with the formula C₆H₈O₇, naturally found in citrus fruits. It is widely used as a food additive for flavoring and preservative purposes, and has the E number E330. It has the CAS Number: 5949-29-1 (for citric acid monohydrate) and IUPAC name: 2-hydroxypropane-1,2,3-tricarboxylic acid.

The term “Gelatin” refers to a protein substance derived from collagen, typically extracted from animal by-products. It has the formula (C₁₇H₃₂N₅O₆) n (where n can range from about 80 to over 3000, depending on the specific gelatin sample and its molecular weight distribution) and is used as a gelling agent in food, pharmaceuticals, and cosmetic products. It has the CAS Number: 9000-70-8.

The term “Green Apple Flavor” or “Green Apple” refers to a synthetic or natural flavoring agent that mimics the tart and crisp taste of green apples. It is commonly used in confectionery, beverages, and baked goods.

The term “Magnasweet MM100” refers to a proprietary sweetness enhancer and flavor modifier derived from licorice root. It is used to intensify sweetness, mask off-notes, and extend the perception of sweetness in food and beverage products. Magnasweet MM100 is manufactured by Mafco Worldwide. Mafco Worldwide has production facilities in multiple locations, including: Mafco Worldwide LLC in Camden, NJ, USA; Shanghai Mafco Biotech Co, Ltd. in Shanghai, China; and ZFTZ Mafco Biotech Co, Ltd. in Zhangjiagang City, Jiangsu Province, China.

The term “Malic Acid” refers to an organic compound with the formula C₄H₆O₅, naturally occurring in fruits and vegetables. It is used as a food additive for flavoring and pH control, with the E number E296. It has the CAS Number: 6915-15-7, IUPAC name: (S)-2-hydroxybutanedioic acid, and INII: 817LIN4CKP.

The term “Mixed Berry Flavor” or “Mixed Berry” refers to a flavoring agent that combines the taste profiles of various berries, typically including strawberry, raspberry, and blueberry. It can be natural or artificial and is used in a wide range of food and beverage products.

The term “Mountain Berry Flavor” or “Mountain Berry” refers to a flavoring agent designed to evoke the taste of berries found in mountainous regions. It often combines flavors of wild berries and may include notes of blueberry, raspberry, and blackberry.

The term “Pectin” refers to a structural heteropolysaccharide contained in the primary cell walls of terrestrial plants. It has the formula (C₆H₁₀O₇)n (where n can range from approximately 100 to over 1000, depending on the specific pectin sample and its molecular weight distribution) and is used as a gelling agent and stabilizer in food, with the E number E440.

The term “Pineapple Flavor” or “Pineapple” refers to a flavoring agent that replicates the sweet and tangy taste of pineapple. It can be natural or artificial and is commonly used in confectionery, beverages, and dairy products.

The term “Potassium Sorbate” refers to a potassium salt of sorbic acid with the chemical formula C₆H₇KO₂. It is widely used as a food preservative to inhibit molds and yeasts, and has the E number E202.

The term “Raspberry Concentrate” or “Raspberry” refers to a concentrated form of raspberry juice, typically made by removing water from fresh raspberry juice. It is used to add intense raspberry flavor to food and beverage products.

The term “Sodium Citrate” refers to the sodium salt of citric acid with the chemical formula Na₃C₆H₅O₇. It is used as a food additive for flavoring and as a preservative, with the E number E331.

The term “Slurry” refers to a mixture of solids that is dispersed, suspended, solubilized, and/or dissolved in liquid. Together, the solids and liquid will include those substances used to manufacture the chewable gummy. The solid substances employed in the manufacture of the chewable gummy can essentially be dissolved in the liquid, can essentially be suspended in the liquid, can essentially be dispersed in the liquid, can essentially be solubilized in the liquid, or a combination thereof. A chewable gummy can be formed by curing a cast slurry. The curing can be carried out, e.g., at an elevated temperature, for a period of time. In doing so, a significant amount of the solvent (e.g., water) may be removed. The remaining water will contribute to the moisture content in the chewable gummy (in addition to any moisture from the surrounding environment that is picked up by any hygroscopic substances employed).

The term “Sugar” refers to a sweet crystalline substance, primarily sucrose (C₁₂H₂₂O₁₁), extracted from sugar cane or sugar beets. It is used as a sweetener and preservative in food and beverage products.

The term “Tapioca Syrup” refers to a sweetener derived from cassava root starch. It is composed of complex carbohydrates and is used as an alternative to corn syrup in food products.

The term “USP Water” refers to water that meets the standards set by the United States Pharmacopeia (USP) for purity and quality. It is used in pharmaceutical and food production where high-quality water is required.

The term “Watermelon Flavor” refers to a flavoring agent that mimics the sweet and refreshing taste of watermelon. It can be natural or artificial and is commonly used in beverages, confectionery, and frozen desserts.

### Specific Ranges, Values, and Embodiments

The specific embodiments describing the ranges and values provided below are for illustration purposes only, and do not otherwise limit the scope of the disclosed subject matter, as defined by the claims.

In specific embodiments, the water is present in the chewable gummy in 15±5 wt. %.

In specific embodiments, the water is present in the chewable gummy in 15±4 wt. %.

In specific embodiments, the water is present in the chewable gummy in 15±3 wt. %.

In specific embodiments, the water is present in the chewable gummy in 15±2 wt. %.

In specific embodiments, the water is present in the chewable gummy in 15±1 wt. %.

In specific embodiments, the gelatin is present in the chewable gummy in 7.39±1 wt. %.

In specific embodiments, the gelatin is present in the chewable gummy in 7.39±0.75 wt. %.

In specific embodiments, the gelatin is present in the chewable gummy in 7.39±0.5 wt. %.

In specific embodiments, the gelatin is present in the chewable gummy in 7.39±0.25 wt. %.

In specific embodiments, the gelatin is present in the chewable gummy in 7.39±0.1 wt. %.

In specific embodiments, the sugar is present in the chewable gummy in 31.66±3 wt. %.

In specific embodiments, the sugar is present in the chewable gummy in 31.66±2.5 wt. %.

In specific embodiments, the sugar is present in the chewable gummy in 31.66±2 wt. %.

In specific embodiments, the sugar is present in the chewable gummy in 31.66±1.5 wt. %.

In specific embodiments, the sugar is present in the chewable gummy in 31.66±1 wt. %.

In specific embodiments, the sugar is present in the chewable gummy in 31.66±0.5 wt. %.

In specific embodiments, the pectin is present in the chewable gummy in 1.06±0.1 wt. %.

In specific embodiments, the pectin is present in the chewable gummy in 1.06±0.075 wt. %.

In specific embodiments, the pectin is present in the chewable gummy in 1.06±0.05 wt. %.

In specific embodiments, the pectin is present in the chewable gummy in 1.06±0.025 wt. %.

In specific embodiments, the pectin is present in the chewable gummy in 1.06±0.01 wt. %.

In specific embodiments, the tapioca syrup is present in the chewable gummy in 23.21±2.5 wt. %.

In specific embodiments, the tapioca syrup is present in the chewable gummy in 23.21±2 wt. %.

In specific embodiments, the tapioca syrup is present in the chewable gummy in 23.21±1.5 wt. %.

In specific embodiments, the tapioca syrup is present in the chewable gummy in 23.21±1 wt. %.

In specific embodiments, the tapioca syrup is present in the chewable gummy in 23.21±0.5 wt. %.

In specific embodiments, the tapioca syrup is present in the chewable gummy in 23.21±0.25 wt. %.

In specific embodiments, the raspberry concentrate is present in the chewable gummy in 7.39±1 wt. %.

In specific embodiments, the raspberry concentrate is present in the chewable gummy in 7.39±0.75 wt. %.

In specific embodiments, the raspberry concentrate is present in the chewable gummy in 7.39±0.5 wt. %.

In specific embodiments, the raspberry concentrate is present in the chewable gummy in 7.39±0.25 wt. %.

In specific embodiments, the raspberry concentrate is present in the chewable gummy in 7.39±0.1 wt. %.

In specific embodiments, the citric acid is present in the chewable gummy in 7.39±0.1 wt. %.

In specific embodiments, the citric acid is present in the chewable gummy in 7.39±0.075 wt. %.

In specific embodiments, the citric acid is present in the chewable gummy in 7.39±0.05 wt. %.

In specific embodiments, the citric acid is present in the chewable gummy in 7.39±0.025 wt. %.

In specific embodiments, the citric acid is present in the chewable gummy in 7.39±0.01 wt. %.

In specific embodiments, the malic acid is present in the chewable gummy in 1.06±1 wt. %.

In specific embodiments, the malic acid is present in the chewable gummy in 1.06±0.75 wt. %.

In specific embodiments, the malic acid is present in the chewable gummy in 1.06±0.5 wt. %.

In specific embodiments, the malic acid is present in the chewable gummy in 1.06±0.25 wt. %.

In specific embodiments, the potassium sorbate is present in the chewable gummy in 0.26±0.025 wt. %.

In specific embodiments, the potassium sorbate is present in the chewable gummy in 0.26±0.020 wt. %.

In specific embodiments, the potassium sorbate is present in the chewable gummy in 0.26±0.015 wt. %.

In specific embodiments, the potassium sorbate is present in the chewable gummy in 0.26±0.010 wt. %.

In specific embodiments, the sodium citrate is present in the chewable gummy in 0.26±0.025 wt. %.

In specific embodiments, the sodium citrate is present in the chewable gummy in 0.26±0.020 wt. %.

In specific embodiments, the sodium citrate is present in the chewable gummy in 0.26±0.015 wt. %.

In specific embodiments, the sodium citrate is present in the chewable gummy in 0.26±0.010 wt. %.

In specific embodiments, the Magnasweet MM100 is present in the chewable gummy in 0.05±0.005 wt. %.

In specific embodiments, the Magnasweet MM100 is present in the chewable gummy in 0.05±0.004 wt. %.

In specific embodiments, the Magnasweet MM100 is present in the chewable gummy in 0.05±0.003 wt. %.

In specific embodiments, the Magnasweet MM100 is present in the chewable gummy in 0.05±0.002 wt. %.

In specific embodiments, the Magnasweet MM100 is present in the chewable gummy in 0.05±0.001 wt. %.

In specific embodiments, the Magnasweet MM100 is present in the chewable gummy in 0.05±0.0005 wt. %.

In specific embodiments, the HMB is present in the chewable gummy in 8.38±2 wt. %.

In specific embodiments, the HMB is present in the chewable gummy in 8.38±1.5 wt. %.

In specific embodiments, the HMB is present in the chewable gummy in 8.38±1 wt. %.

In specific embodiments, the HMB is present in the chewable gummy in 8.38±0.75 wt. %.

In specific embodiments, the HMB is present in the chewable gummy in 8.38±0.5 wt. %.

In specific embodiments, the HMB is present in the chewable gummy in 8.38±0.25 wt. %.

In specific embodiments, the HMB is present in the chewable gummy in 8.38±0.1 wt. %.

In specific embodiments, the flavoring is present in the chewable gummy in a combined amount of 5.5±1 wt. %.

In specific embodiments, the flavoring is present in the chewable gummy in a combined amount of 5±1 wt. %.

In specific embodiments, the flavoring is present in the chewable gummy in a combined amount of 4.5±1 wt. %.

In specific embodiments, the flavoring is present in the chewable gummy in a combined amount of 4.5±0.75 wt. %.

In specific embodiments, the flavoring is present in the chewable gummy in a combined amount of 4.5±0.5 wt. %.

In specific embodiments, the flavoring is present in the chewable gummy in a combined amount of 4.5±0.25 wt. %.

In specific embodiments, the flavoring is present in the chewable gummy in a combined amount of 4±1 wt. %.

In specific embodiments, the flavoring is present in the chewable gummy in a combined amount of 3.5±1 wt. %.

In specific embodiments, the flavoring is present in the chewable gummy in a combined amount of 3±1 wt. %.

In specific embodiments, the flavoring is present in the chewable gummy in a combined amount of 2.5±1 wt. %.

In specific embodiments, the flavoring is present in the chewable gummy in a combined amount of 2.5±0.75 wt. %.

In specific embodiments, the flavoring is present in the chewable gummy in a combined amount of 2.5±0.5 wt. %.

In specific embodiments, the flavoring is present in the chewable gummy in a combined amount of 2.5±0.25 wt. %.

In specific embodiments, the mountain berry is present in the chewable gummy.

In specific embodiments, the mountain berry is not present in the chewable gummy.

In specific embodiments, the mountain berry is present in the chewable gummy in 2.38±2.38 wt. %.

In specific embodiments, the mountain berry is present in the chewable gummy in 2.38±2 wt. %.

In specific embodiments, the mountain berry is present in the chewable gummy in 2.38±1.5 wt. %.

In specific embodiments, the mountain berry is present in the chewable gummy in 2.38±1 wt. %.

In specific embodiments, the mountain berry is present in the chewable gummy in 2.38±0.5 wt. %.

In specific embodiments, the mountain berry is present in the chewable gummy in 2.38±0.25 wt. %.

In specific embodiments, the mountain berry is present in the chewable gummy in 2.38±0.1 wt. %.

In specific embodiments, the mixed berry is present in the chewable gummy.

In specific embodiments, the mixed berry is not present in the chewable gummy.

In specific embodiments, the mixed berry is present in the chewable gummy in 2.38±2.38 wt. %.

In specific embodiments, the mixed berry is present in the chewable gummy in 2.38±2 wt. %.

In specific embodiments, the mixed berry is present in the chewable gummy in 2.38±1.5 wt. %.

In specific embodiments, the mixed berry is present in the chewable gummy in 2.38±1 wt. %.

In specific embodiments, the mixed berry is present in the chewable gummy in 2.38±0.5 wt. %.

In specific embodiments, the mixed berry is present in the chewable gummy in 2.38±0.25 wt. %.

In specific embodiments, the mixed berry is present in the chewable gummy in 2.38±0.1 wt. %.

In specific embodiments, the watermelon flavor is present in the chewable gummy.

In specific embodiments, the watermelon flavor is not present in the chewable gummy.

In specific embodiments, the watermelon flavor is present in the chewable gummy in 4.75±4 wt. %.

In specific embodiments, the watermelon flavor is present in the chewable gummy in 4.75±3.75 wt. %.

In specific embodiments, the watermelon flavor is present in the chewable gummy in 4.75±3.5 wt. %.

In specific embodiments, the watermelon flavor is present in the chewable gummy in 4.75±3.25 wt. %.

In specific embodiments, the watermelon flavor is present in the chewable gummy in 4.75±3 wt. %.

In specific embodiments, the watermelon flavor is present in the chewable gummy in 4.75±2.75 wt. %.

In specific embodiments, the watermelon flavor is present in the chewable gummy in 4.75±2.5 wt. %.

In specific embodiments, the watermelon flavor is present in the chewable gummy in 4.75±2.25 wt. %.

In specific embodiments, the watermelon flavor is present in the chewable gummy in 4.75±2 wt. %.

In specific embodiments, the watermelon flavor is present in the chewable gummy in 4.75±1.75 wt. %.

In specific embodiments, the watermelon flavor is present in the chewable gummy in 4.75±1.5 wt. %.

In specific embodiments, the watermelon flavor is present in the chewable gummy in 4.75±1.25 wt. %.

In specific embodiments, the watermelon flavor is present in the chewable gummy in 4.75±1 wt. %.

In specific embodiments, the green apple is present in the chewable gummy.

In specific embodiments, the green apple is not present in the chewable gummy.

In specific embodiments, the green apple is present in the chewable gummy in 2.32±2.32 wt. %.

In specific embodiments, the green apple is present in the chewable gummy in 2.32±2 wt. %.

In specific embodiments, the green apple is present in the chewable gummy in 2.32±1.5 wt. %.

In specific embodiments, the green apple is present in the chewable gummy in 2.32±1 wt. %.

In specific embodiments, the green apple is present in the chewable gummy in 2.32±0.75 wt. %.

In specific embodiments, the green apple is present in the chewable gummy in 2.32±0.5 wt. %.

In specific embodiments, the green apple is present in the chewable gummy in 2.32±0.25 wt. %.

In specific embodiments, the green apple is present in the chewable gummy in 2.32±0.1 wt. %.

In specific embodiments, the acid flavor reducer is present in the chewable gummy in 0.40±0.05 wt. %.

In specific embodiments, the acid flavor reducer is present in the chewable gummy in 0.40±0.04 wt. %.

In specific embodiments, the acid flavor reducer is present in the chewable gummy in 0.40±0.03 wt. %.

In specific embodiments, the acid flavor reducer is present in the chewable gummy in 0.40±0.02 wt. %.

In specific embodiments, the acid flavor reducer is present in the chewable gummy in 0.40±0.01 wt. %.

In specific embodiments, the chewable gummy includes:

| Ingredient | Amount (wt. %) | Amount (g) |
|---|---|---|
| USP Water | 13.47 ± 1.5 | 0.81 ± 0.1 |
| Gelatin | 7.39 ± 0.8 | 0.44 ± 0.05 |
| Sugar | 31.66 ± 4 | 1.90 ± 0.2 |
| Pectin | 1.06 ± 0.2 | 0.06 ± 0.01 |
| Tapioca Syrup | 23.21 ± 3 | 1.39 ± 0.2 |
| Raspberry Concentrate | 7.39 ± 1 | 0.44 ± 0.05 |
| Citric Acid | 1.06 ± 0.2 | 0.06 ± 0.01 |
| Malic Acid | 0.66 ± 0.1 | 0.04 ± 0.01 |
| Potassium Sorbate | 0.26 ± 0.05 | 0.02 ± 0.005 |
| Sodium Citrate | 0.26 ± 0.05 | 0.02 ± 0.005 |
| Magnasweet MM100 | 0.05 ± 0.01 | 0.003 ± 0.001 |
| HMB | 8.38 ± 1 | 0.50 ± 0.05 |
| Mountain Berry | 2.38 ± 0.3 | 0.14 ± 0.02 |
| Mixed Berry | 2.38 ± 0.3 | 0.14 ± 0.02 |
| Acid Flavor Reducer | 0.40 ± 0.05 | 0.02 ± 0.003 |
| Total | 100 | 6 ± 0.75 |

In specific embodiments, the chewable gummy includes:

| Ingredient | Amount (wt. %) | Amount (g) |
|---|---|---|
| USP Water | 13.47 | 0.81 |
| Gelatin | 7.39 | 0.44 |
| Sugar | 31.66 | 1.90 |
| Pectin | 1.06 | 0.06 |
| Tapioca Syrup | 23.21 | 1.39 |
| Raspberry Concentrate | 7.39 | 0.44 |
| Citric Acid | 1.06 | 0.06 |
| Malic Acid | 0.66 | 0.04 |
| Potassium Sorbate | 0.26 | 0.02 |
| Sodium Citrate | 0.26 | 0.02 |
| Magnasweet MM100 | 0.05 | 0.003 |
| HMB | 8.38 | 0.50 |
| Mountain Berry | 2.38 | 0.14 |
| Mixed Berry | 2.38 | 0.14 |
| Acid Flavor Reducer | 0.40 | 0.02 |
| Total | 100 | 6 |

In specific embodiments, the chewable gummy includes:

| Ingredient | Amount (wt. %) | Amount (g) |
|---|---|---|
| USP Water | 13.47 ± 1.5 | 0.81 ± 0.1 |
| Gelatin | 7.39 ± 1 | 0.44 ± 0.1 |
| Sugar | 31.66 ± 4 | 1.90 ± 0.3 |
| Pectin | 1.06 ± 0.2 | 0.06 ± 0.01 |
| Tapioca Syrup | 23.21 ± 3 | 1.40 ± 0.3 |
| Raspberry Concentrate | 7.39 ± 1 | 0.44 ± 0.05 |
| Citric Acid | 1.06 ± 0.2 | 0.06 ± 0.01 |
| Malic Acid | 0.67 ± 0.1 | 0.04 ± 0.01 |
| Potassium Sorbate | 0.26 ± 0.05 | 0.02 ± 0.005 |
| Sodium Citrate | 0.26 ± 0.05 | 0.02 ± 0.005 |
| Magnasweet MM100 | 0.05 ± 0.01 | 0.003 ± 0.001 |
| HMB | 8.38 ± 1 | 0.50 ± 0.1 |
| Watermelon Flavor | 4.75 ± 0.5 | 0.28 ± 0.05 |
| Acid Flavor Reducer | 0.40 ± 0.05 | 0.02 ± 0.005 |
| Total | 100 | 6 ± 0.75 |

In specific embodiments, the chewable gummy includes:

| Ingredient | Amount (wt. %) | Amount (g) |
|---|---|---|
| USP Water | 13.47 | 0.81 |
| Gelatin | 7.39 | 0.44 |
| Sugar | 31.66 | 1.90 |
| Pectin | 1.06 | 0.06 |
| Tapioca Syrup | 23.21 | 1.40 |
| Raspberry Concentrate | 7.39 | 0.44 |
| Citric Acid | 1.06 | 0.06 |
| Malic Acid | 0.67 | 0.04 |
| Potassium Sorbate | 0.26 | 0.02 |
| Sodium Citrate | 0.26 | 0.02 |
| Magnasweet MM100 | 0.05 | 0.003 |
| HMB | 8.38 | 0.50 |
| Watermelon Flavor | 4.75 | 0.28 |
| Acid Flavor Reducer | 0.40 | 0.02 |
| Total | 100 | 6 |

In specific embodiments, the chewable gummy includes:

| Ingredient | Amount (wt. %) | Amount (g) |
|---|---|---|
| USP Water | 15.90 ± 1.5 | 0.95 ± 0.1 |
| Gelatin | 7.39 ± 1 | 0.44 ± 0.1 |
| Sugar | 31.66 ± 3 | 1.90 ± 0.2 |
| Pectin | 1.06 ± 0.1 | 0.06 ± 0.01 |
| Tapioca Syrup | 23.22 ± 3 | 1.40 ± 0.2 |
| Raspberry Concentrate | 7.39 ± 1 | 0.44 ± 0.05 |
| Citric Acid | 1.06 ± 0.1 | 0.06 ± 0.01 |
| Malic Acid | 0.66 ± 0.1 | 0.04 ± 0.01 |
| Potassium Sorbate | 0.26 ± 0.03 | 0.02 ± 0.005 |
| Sodium Citrate | 0.26 ± 0.03 | 0.02 ± 0.005 |
| Magnasweet MM100 | 0.05 ± 0.01 | 0.003 ± 0.001 |
| HMB | 8.38 ± 1 | 0.50 ± 0.1 |
| Pineapple Flavor | 2.32 ± 0.3 | 0.14 ± 0.02 |
| Acid Flavor Reducer | 0.40 ± 0.1 | 0.02 ± 0.005 |
| Total | 100 | 6 ± 0.75 |

In specific embodiments, the chewable gummy includes:

| Ingredient | Amount (wt. %) | Amount (g) |
|---|---|---|
| USP Water | 15.90 | 0.95 |
| Gelatin | 7.39 | 0.44 |
| Sugar | 31.66 | 1.90 |
| Pectin | 1.06 | 0.06 |
| Tapioca Syrup | 23.22 | 1.40 |
| Raspberry Concentrate | 7.39 | 0.44 |
| Citric Acid | 1.06 | 0.06 |
| Malic Acid | 0.66 | 0.04 |
| Potassium Sorbate | 0.26 | 0.02 |
| Sodium Citrate | 0.26 | 0.02 |
| Magnasweet MM100 | 0.05 | 0.003 |
| HMB | 8.38 | 0.50 |
| Pineapple Flavor | 2.32 | 0.14 |
| Acid Flavor Reducer | 0.40 | 0.02 |
| Total | 100 | 6 |

In specific embodiments, the chewable gummy includes:

| Ingredient | Amount (wt. %) | Amount (g) |
|---|---|---|
| USP Water | 15.90 ± 1.5 | 0.95 ± 0.1 |
| Gelatin | 7.39 ± 1 | 0.44 ± 0.05 |
| Sugar | 31.66 ± 3 | 1.90 ± 0.2 |
| Pectin | 1.06 ± 0.1 | 0.06 ± 0.01 |
| Tapioca Syrup | 23.22 ± 3 | 1.40 ± 0.2 |
| Raspberry Concentrate | 7.39 ± 1 | 0.44 ± 0.1 |
| Citric Acid | 1.06 ± 0.1 | 0.06 ± 0.01 |
| Malic Acid | 0.66 ± 0.1 | 0.04 ± 0.01 |
| Potassium Sorbate | 0.26 ± 0.05 | 0.02 ± 0.005 |
| Sodium Citrate | 0.26 ± 0.05 | 0.02 ± 0.005 |
| Magnasweet MM100 | 0.05 ± 0.01 | 0.003 ± 0.001 |
| HMB | 8.38 ± 1 | 0.50 ± 0.05 |
| Green Apple | 2.32 ± 0.3 | 0.14 ± 0.02 |
| Acid Flavor Reducer | 0.40 ± 0.1 | 0.02 ± 0.002 |
| Total | 100 | 6 ± 0.75 |

In specific embodiments, the chewable gummy includes:

| Ingredient | Amount (wt. %) | Amount (g) |
|---|---|---|
| USP Water | 15.90 | 0.95 |
| Gelatin | 7.39 | 0.44 |
| Sugar | 31.66 | 1.90 |
| Pectin | 1.06 | 0.06 |
| Tapioca Syrup | 23.22 | 1.40 |
| Raspberry Concentrate | 7.39 | 0.44 |
| Citric Acid | 1.06 | 0.06 |
| Malic Acid | 0.66 | 0.04 |
| Potassium Sorbate | 0.26 | 0.02 |
| Sodium Citrate | 0.26 | 0.02 |
| Magnasweet MM100 | 0.05 | 0.003 |
| HMB | 8.38 | 0.50 |
| Green Apple | 2.32 | 0.14 |
| Acid Flavor Reducer | 0.40 | 0.02 |
| Total | 100 | 6 |

In specific embodiments, the chewable gummy has a mass of 6±3 g.

In specific embodiments, the chewable gummy has a mass of 6±2.5 g.

In specific embodiments, the chewable gummy has a mass of 6±2 g.

In specific embodiments, the chewable gummy has a mass of 6±1.5 g.

In specific embodiments, the chewable gummy has a mass of 6±1 g.

In specific embodiments, the chewable gummy has a mass of 6±0.5 g.

In specific embodiments, the chewable gummy has a mass of 6±0.25 g.

In specific embodiments, the chewable gummy has a pH of 4.51±0.5.

In specific embodiments, the chewable gummy has a pH of 4.51±0.25.

In specific embodiments, the chewable gummy has a pH of 4.51±0.1.

In specific embodiments, the chewable gummy has a pH of 4.51±0.05.

In specific embodiments, the chewable gummy has a Brix of 72±7.

In specific embodiments, the chewable gummy has a Brix of 72±6.

In specific embodiments, the chewable gummy has a Brix of 72±5.

In specific embodiments, the chewable gummy has a Brix of 72±4.

In specific embodiments, the chewable gummy has a Brix of 72±3.

In specific embodiments, the chewable gummy has a Brix of 72±2.

In specific embodiments, the chewable gummy has a Brix of 72±1.

In specific embodiments, the chewable gummy has a total water content of 10-20 wt. %.

In specific embodiments, the chewable gummy has a total water content of 12-17 wt. %.

In specific embodiments, the chewable gummy has a total water content of 12.5-15.5 wt. %.

In specific embodiments, the chewable gummy has a total water content of 13-16 wt. %.

In specific embodiments, the chewable gummy is configured as a dietary supplement.

In specific embodiments, the chewable gummy is configured as a medical food.

In specific embodiments, the chewable gummy is orally administered to a human subject.

In specific embodiments, the chewable gummy is orally administered to a human subject to promote wound healing to the human subject following surgery or injury.

In specific embodiments, the chewable gummy is orally administered to a human subject to provide nutritional support to the human subject.

In specific embodiments, the chewable gummy is orally administered to a human subject to provide nutritional support to the human subject with muscle wasting due to cancer or HIV/AIDS.

In specific embodiments, the chewable gummy is orally administered to a human subject to (a) increase exercise-induced gains in muscle size, muscle strength, and/or lean body mass, (b) reduce skeletal muscle damage from exercise, (c) improve aerobic exercise performance, and/or (d) expedite recovery from exercise.

In specific embodiments, the chewable gummy is orally administered to a human subject to preserve and/or increase lean body mass and muscle strength in the human subject experiencing age-related muscle loss.

In specific embodiments, the chewable gummy is orally administered to a human subject to preserve lean body mass in muscle wasting conditions.

In specific embodiments, the chewable gummy is orally administered to a human subject to preserve lean body mass in human subjects afflicted with sarcopenia, or is at risk thereof.

In specific embodiments, the chewable gummy is orally administered to a human subject to prevent or mitigate muscle atrophy during bed rest.

In specific embodiments, the chewable gummy is orally administered to a human subject to reduce the loss of muscle mass, muscle function, and muscle strength that occurs in hypercatabolic disease states, such as cancer cachexia.

In specific embodiments, the chewable gummy is orally administered to a human subject as an adjunct therapy in conjunction with resistance exercise.

In specific embodiments, the chewable gummy is orally administered to a human subject as an adjunct therapy in conjunction with resistance exercise and consumption of a high-protein diet.

In specific embodiments, the chewable gummy is orally administered to a human subject to (a) increase exercise-induced gains in muscle size, muscle strength and power, (b) increase lean body mass, (c) reduce exercise-induced skeletal muscle damage, and/or (d) expedite recovery from high-intensity exercise.

In specific embodiments, the chewable gummy is orally administered to a human subject to improve aerobic exercise performance and increases gains in aerobic fitness when combined with high-intensity interval training.

In specific embodiments, the chewable gummy is orally administered to a human subject to reduce skeletal muscle damage from prolonged or high-intensity exercise.

In specific embodiments, the chewable gummy is orally administered to a human subject, such that 3-6 grams of HMB per day are orally administered to the human subject.

In specific embodiments, the chewable gummy is orally administered to a human subject, such that 1-3 chewable gummies are orally administered to the human subject 0.25-2.5 hours prior to exercise.

In specific embodiments, the chewable gummy is orally administered to a human subject, such that 1-3 chewable gummies are orally administered to the human subject 0.5-2 hours prior to exercise.

In specific embodiments, the chewable gummy is orally administered to a human subject, such that a total of 1-15 chewable gummies are orally administered to the human subject throughout the day.

In specific embodiments, the chewable gummy is orally administered to a human subject, such that a total of 1-10 chewable gummies are orally administered to the human subject throughout the day.

In specific embodiments, the chewable gummy is orally administered to a human subject, such that a total of 1-5 chewable gummies are orally administered to the human subject throughout the day.

In specific embodiments, the chewable gummy is orally administered to a human subject, such that a total of 2-5 chewable gummies are orally administered to the human subject throughout the day.

All publications, patents, and patent applications referred to herein are incorporated by reference in their entirety to the same extent as if each individual publication, patent, or patent application was specifically and individually indicated to be incorporated by reference in its entirety.

The following examples are set forth to further illustrate the presently disclosed subject matter (e.g., chewable gummies, methods of using the same, and methods of preparing the same). The below examples, however, should not be construed as limiting in any manner the present invention, as set forth in the claims.

### Enumerated Embodiments

Specific enumerated embodiments <1> to <36> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

<Embodiment 1> A chewable gummy comprising:
β-hydroxy β-methylbutyric acid (HMB);
gelling agent comprising at least one of gelatin and pectin;
sweetening agent comprising at least one of sugar, tapioca syrup, and magnasweet MM100;
flavoring agent comprising at least one of raspberry flavor, green apple flavor, mountain berry flavor, mixed berry flavor, pineapple flavor, watermelon flavor, citric acid, malic acid, and acid flavor reducer;
preservative comprising at least one of potassium sorbate and sodium citrate, and solvent comprising water;
   wherein,
the chewable gummy is configured as a unit dose having a mass of 6±1 g;
the pH is 4.51±0.5;
the Brix is 72±7; and
the water content is 12-17 wt. %.

<Embodiment 2> The chewable gummy of embodiment <1>, wherein the β-hydroxy β-methylbutyric acid (HMB) is in the free acid form, β-hydroxy β-methylbutyric acid (HMB-FA).

<Embodiment 3> The chewable gummy of embodiment <1>, wherein the β-hydroxy β-methylbutyric acid (HMB) is in the monohydrated calcium salt of the conjugate base form, calcium β-hydroxy β-methylbutyrate monohydrate (HMB-Ca, CaHMB).

<Embodiment 4> The chewable gummy of any one of embodiments <1> to <3>, wherein the β-hydroxy β-methylbutyric acid (HMB) is present in 8.3±1 wt. %.

<Embodiment 5> The chewable gummy of any one of embodiments <1> to <3>, wherein the β-hydroxy β-methylbutyric acid (HMB) is present in 0.5±0.05 g.

<Embodiment 6> The chewable gummy of any one of embodiments <1> to <5>, wherein the gelling agent comprises gelatin and pectin.

<Embodiment 7> The chewable gummy of any one of embodiments <1> to <5>, wherein the gelling agent comprises gelatin and pectin, present in a combined amount of 0.5±0.05 g.

<Embodiment 8> The chewable gummy of any one of embodiments <1> to <5>, wherein the gelling agent comprises gelatin and pectin, present in a combined amount of 8.4±0.8 wt. %.

<Embodiment 9> The chewable gummy of any one of embodiments <1> to <8>, wherein the sweetening agent comprises sugar, tapioca syrup, and magnasweet MM100.

<Embodiment 10> The chewable gummy of any one of embodiments <1> to <9>, wherein the sweetening agent comprises sugar, tapioca syrup, and magnasweet MM100, present in a combined amount of 3.3±0.5 g.

<Embodiment 11> The chewable gummy of any one of embodiments <1> to <10>, wherein the sweetening agent comprises sugar, tapioca syrup, and magnasweet MM100, present in a combined amount of 54.9±5 wt. %.

<Embodiment 12> The chewable gummy of any one of embodiments <1> to <11>, wherein the flavoring agent comprises citric acid, malic acid, and acid flavor reducer, present in a combined amount of 0.13±0.01 g.

<Embodiment 13> The chewable gummy of any one of embodiments <1> to <11>, wherein the flavoring agent comprises citric acid, malic acid, and acid flavor reducer, present in a combined amount of 2.1±0.2 wt. %.

<Embodiment 14> The chewable gummy of any one of embodiments <1> to <13>, wherein the flavoring agent comprises raspberry flavor, mountain berry flavor, and mixed berry flavor.

<Embodiment 15> The chewable gummy of any one of embodiments <1> to <13>, wherein the flavoring agent comprises raspberry flavor and watermelon flavor.

<Embodiment 16> The chewable gummy of any one of embodiments <1> to <13>, wherein the flavoring agent comprises raspberry flavor and pineapple flavor.

<Embodiment 17> The chewable gummy of any one of embodiments <1> to <13>, wherein the flavoring agent comprises raspberry flavor and green apple flavor.

<Embodiment 18> The chewable gummy of any one of embodiments <1> to <17>, wherein the water content is 13-16 wt. %.

<Embodiment 19> The chewable gummy of any one of embodiments <1> to <18>, wherein the pH is 4.51±0.25.

<Embodiment 20> The chewable gummy of any one of embodiments <1> to <19>, wherein the Brix is 72±5.

<Embodiment 21> The chewable gummy of any one of embodiments <1> to <20>, comprising:

| Ingredient | Amount (wt. %) | Amount (g) |
|---|---|---|
| USP Water | 13.47 ± 1.5 | 0.81 ± 0.1 |
| Gelatin | 7.39 ± 0.8 | 0.44 ± 0.05 |
| Sugar | 31.66 ± 4 | 1.90 ± 0.2 |
| Pectin | 1.06 ± 0.2 | 0.06 ± 0.01 |
| Tapioca Syrup | 23.21 ± 3 | 1.39 ± 0.2 |
| Raspberry Concentrate | 7.39 ± 1 | 0.44 ± 0.05 |
| Citric Acid | 1.06 ± 0.2 | 0.06 ± 0.01 |
| Malic Acid | 0.66 ± 0.1 | 0.04 ± 0.01 |
| Potassium Sorbate | 0.26 ± 0.05 | 0.02 ± 0.005 |
| Sodium Citrate | 0.26 ± 0.05 | 0.02 ± 0.005 |
| Magnasweet MM100 | 0.05 ± 0.01 | 0.003 ± 0.001 |
| HMB | 8.38 ± 1 | 0.50 ± 0.05 |
| Mountain Berry | 2.38 ± 0.3 | 0.14 ± 0.02 |
| Mixed Berry | 2.38 ± 0.3 | 0.14 ± 0.02 |
| Acid Flavor Reducer | 0.40 ± 0.05 | 0.02 ± 0.003 |
| Total | 100 | 6 ± 0.75 |

<Embodiment 22> The chewable gummy of embodiments <1> to <20>, comprising:

| Ingredient | Amount (wt. %) | Amount (g) |
|---|---|---|
| USP Water | 13.47 | 0.81 |
| Gelatin | 7.39 | 0.44 |
| Sugar | 31.66 | 1.90 |
| Pectin | 1.06 | 0.06 |
| Tapioca Syrup | 23.21 | 1.39 |
| Raspberry Concentrate | 7.39 | 0.44 |
| Citric Acid | 1.06 | 0.06 |
| Malic Acid | 0.66 | 0.04 |
| Potassium Sorbate | 0.26 | 0.02 |
| Sodium Citrate | 0.26 | 0.02 |
| Magnasweet MM100 | 0.05 | 0.003 |
| HMB | 8.38 | 0.50 |
| Mountain Berry | 2.38 | 0.14 |
| Mixed Berry | 2.38 | 0.14 |
| Acid Flavor Reducer | 0.40 | 0.02 |
| Total | 100 | 6 |

<Embodiment 23> The chewable gummy of embodiments <1> to <20>, comprising:

| Ingredient | Amount (wt. %) | Amount (g) |
|---|---|---|
| USP Water | 13.47 ± 1.5 | 0.81 ± 0.1 |
| Gelatin | 7.39 ± 1 | 0.44 ± 0.1 |
| Sugar | 31.66 ± 4 | 1.90 ± 0.3 |
| Pectin | 1.06 ± 0.2 | 0.06 ± 0.01 |
| Tapioca Syrup | 23.21 ± 3 | 1.40 ± 0.3 |
| Raspberry Concentrate | 7.39 ± 1 | 0.44 ± 0.05 |
| Citric Acid | 1.06 ± 0.2 | 0.06 ± 0.01 |
| Malic Acid | 0.67 ± 0.1 | 0.04 ± 0.01 |
| Potassium Sorbate | 0.26 ± 0.05 | 0.02 ± 0.005 |
| Sodium Citrate | 0.26 ± 0.05 | 0.02 ± 0.005 |
| Magnasweet MM100 | 0.05 ± 0.01 | 0.003 ± 0.001 |
| HMB | 8.38 ± 1 | 0.50 ± 0.1 |
| Watermelon Flavor | 4.75 ± 0.5 | 0.28 ± 0.05 |
| Acid Flavor Reducer | 0.40 ± 0.05 | 0.02 ± 0.005 |
| Total | 100 | 6 ± 0.75 |

<Embodiment 24> The chewable gummy of embodiments <1> to <20>, comprising:

| Ingredient | Amount (wt. %) | Amount (g) |
|---|---|---|
| USP Water | 13.47 | 0.81 |
| Gelatin | 7.39 | 0.44 |
| Sugar | 31.66 | 1.90 |
| Pectin | 1.06 | 0.06 |
| Tapioca Syrup | 23.21 | 1.40 |
| Raspberry Concentrate | 7.39 | 0.44 |
| Citric Acid | 1.06 | 0.06 |
| Malic Acid | 0.67 | 0.04 |
| Potassium Sorbate | 0.26 | 0.02 |
| Sodium Citrate | 0.26 | 0.02 |
| Magnasweet MM100 | 0.05 | 0.003 |
| HMB | 8.38 | 0.50 |
| Watermelon Flavor | 4.75 | 0.28 |
| Acid Flavor Reducer | 0.40 | 0.02 |
| Total | 100 | 6 |

<Embodiment 25> The chewable gummy of embodiments <1> to <20>, comprising:

| Ingredient | Amount (wt. %) | Amount (g) |
|---|---|---|
| USP Water | 15.90 ± 1.5 | 0.95 ± 0.1 |
| Gelatin | 7.39 ± 1 | 0.44 ± 0.1 |
| Sugar | 31.66 ± 3 | 1.90 ± 0.2 |
| Pectin | 1.06 ± 0.1 | 0.06 ± 0.01 |
| Tapioca Syrup | 23.22 ± 3 | 1.40 ± 0.2 |
| Raspberry Concentrate | 7.39 ± 1 | 0.44 ± 0.05 |
| Citric Acid | 1.06 ± 0.1 | 0.06 ± 0.01 |
| Malic Acid | 0.66 ± 0.1 | 0.04 ± 0.01 |
| Potassium Sorbate | 0.26 ± 0.03 | 0.02 ± 0.005 |
| Sodium Citrate | 0.26 ± 0.03 | 0.02 ± 0.005 |
| Magnasweet MM100 | 0.05 ± 0.01 | 0.003 ± 0.001 |
| HMB | 8.38 ± 1 | 0.50 ± 0.1 |
| Pineapple Flavor | 2.32 ± 0.3 | 0.14 ± 0.02 |
| Acid Flavor Reducer | 0.40 ± 0.1 | 0.02 ± 0.005 |
| Total | 100 | 6 ± 0.75 |

<Embodiment 26> The chewable gummy of embodiments <1> to <20>, comprising:

| Ingredient | Amount (wt. %) | Amount (g) |
|---|---|---|
| USP Water | 15.90 | 0.95 |
| Gelatin | 7.39 | 0.44 |
| Sugar | 31.66 | 1.90 |
| Pectin | 1.06 | 0.06 |
| Tapioca Syrup | 23.22 | 1.40 |
| Raspberry Concentrate | 7.39 | 0.44 |
| Citric Acid | 1.06 | 0.06 |
| Malic Acid | 0.66 | 0.04 |
| Potassium Sorbate | 0.26 | 0.02 |
| Sodium Citrate | 0.26 | 0.02 |
| Magnasweet MM100 | 0.05 | 0.003 |
| HMB | 8.38 | 0.50 |
| Pineapple Flavor | 2.32 | 0.14 |
| Acid Flavor Reducer | 0.40 | 0.02 |
| Total | 100 | 6 |

<Embodiment 27> The chewable gummy of embodiments <1> to <20>, comprising:

| Ingredient | Amount (wt. %) | Amount (g) |
|---|---|---|
| USP Water | 15.90 ± 1.5 | 0.95 ± 0.1 |
| Gelatin | 7.39 ± 1 | 0.44 ± 0.05 |
| Sugar | 31.66 ± 3 | 1.90 ± 0.2 |
| Pectin | 1.06 ± 0.1 | 0.06 ± 0.01 |
| Tapioca Syrup | 23.22 ± 3 | 1.40 ± 0.2 |
| Raspberry Concentrate | 7.39 ± 1 | 0.44 ± 0.1 |
| Citric Acid | 1.06 ± 0.1 | 0.06 ± 0.01 |
| Malic Acid | 0.66 ± 0.1 | 0.04 ± 0.01 |
| Potassium Sorbate | 0.26 ± 0.05 | 0.02 ± 0.005 |
| Sodium Citrate | 0.26 ± 0.05 | 0.02 ± 0.005 |
| Magnasweet MM100 | 0.05 ± 0.01 | 0.003 ± 0.001 |
| HMB | 8.38 ± 1 | 0.50 ± 0.05 |
| Green Apple | 2.32 ± 0.3 | 0.14 ± 0.02 |
| Acid Flavor Reducer | 0.40 ± 0.1 | 0.02 ± 0.002 |
| Total | 100 | 6 ± 0.75 |

<Embodiment 28> The chewable gummy of embodiments <1> to <20>, comprising:

| Ingredient | Amount (wt. %) | Amount (g) |
|---|---|---|
| USP Water | 15.90 | 0.95 |
| Gelatin | 7.39 | 0.44 |
| Sugar | 31.66 | 1.90 |
| Pectin | 1.06 | 0.06 |
| Tapioca Syrup | 23.22 | 1.40 |
| Raspberry Concentrate | 7.39 | 0.44 |
| Citric Acid | 1.06 | 0.06 |
| Malic Acid | 0.66 | 0.04 |
| Potassium Sorbate | 0.26 | 0.02 |
| Sodium Citrate | 0.26 | 0.02 |
| Magnasweet MM100 | 0.05 | 0.003 |
| HMB | 8.38 | 0.50 |
| Green Apple | 2.32 | 0.14 |
| Acid Flavor Reducer | 0.40 | 0.02 |
| Total | 100 | 6 |

<Embodiment 29> The chewable gummy of any one of embodiments <1> to <28> configured as a dietary supplement.

<Embodiment 30> The chewable gummy of any one of embodiments <1> to <28>, configured as a medical food.

<Embodiment 31> A method comprising orally administering the chewable gummy of any one of embodiments <1> to <30> to a human subject.

<Embodiment 32> The method of embodiment <31>:
which promotes wound healing to the human subject following surgery or injury;
which provides nutritional support to the human subject;
which provides nutritional support to the human subject with muscle wasting due to cancer or HIV/AIDS;
which (a) increases exercise-induced gains in muscle size, muscle strength, and/or lean body mass, (b) reduces skeletal muscle damage from exercise, (c) improves aerobic exercise performance, and/or (d) expedites recovery from exercise;
which preserves and/or increases lean body mass and muscle strength in the human subject experiencing age-related muscle loss;
which preserves lean body mass in muscle wasting conditions;
which preserves lean body mass in human subjects afflicted with sarcopenia, or is at risk thereof;
which prevents or mitigates muscle atrophy during bed rest;
which reduces the loss of muscle mass, muscle function, and muscle strength that occurs in hypercatabolic disease states, such as cancer cachexia;
which is an adjunct therapy in conjunction with resistance exercise;
which is an adjunct therapy in conjunction with resistance exercise and consumption of a high-protein diet;
which (a) increases exercise-induced gains in muscle size, muscle strength and power, (b) increases lean body mass, (c) reduces exercise-induced skeletal muscle damage, and/or (d) expedites recovery from high-intensity exercise;
which improves aerobic exercise performance and increases gains in aerobic fitness when combined with high-intensity interval training; and/or
which reduces skeletal muscle damage from prolonged or high-intensity exercise.

<Embodiment 33> The method of any one of embodiments <31> to <32>, in which 3-6 grams of HMB per day are orally administered to the human subject.

<Embodiment 34> The method of any one of embodiments <31> to <33>, wherein 1-3 chewable gummies are orally administered to the human subject 0.25-2.5 hours prior to exercise.

<Embodiment 35> The method of any one of embodiments <31> to <33>, wherein 1-3 chewable gummies are orally administered to the human subject 0.5-2 hours prior to exercise.

<Embodiment 36> The method of any one of embodiments <31> to <33>, in which a total of 1-15 chewable gummies are orally administered to the human subject throughout the day.

### EXAMPLES

### Example 1. Chewable Gummy Containing β-Hydroxy β-Methylbutyric Acid (HMB), Formulation

### Example 1A. Raspberry Flavor

| | | Slurry | | Final Product | |
|---|---|---|---|---|---|
| | | Formula | Formula | Formula | Formula |
| | Ingredient | (wt. %) | (g) | (wt. %) | (g) |
| Total Water | USP Water | 18 | 180.1 | 13.467 | 0.808021108 |
| Phase A | Water | — | 102.92715 | | |
| (Gelatin | Gelatin | 7.00 | 70 | 7.388 | 0.443271768 |
| Solution) | | | | | |
| Phase B | Sugar | 30.00 | 300 | 31.662 | 1.899736148 |
| (Sugar | Pectin | 1.00 | 10 | 1.055 | 0.063324538 |
| Solution) | USP Water | — | 77.17285 | | |
| | Tapioca | 22.00 | 220 | 23.219 | 1.393139842 |
| | Syrup | | | | |
| | Raspberry | 7.00 | 70 | 7.388 | 0.443271768 |
| | Concentrate | | | | |
| Solids (Acids/ | Citric Acid | 1.00 | 10 | 1.055 | 0.063324538 |
| Preservatives) | Malic Acid | 0.625 | 6.25 | 0.660 | 0.039577836 |
| | Potassium | 0.250 | 2.5 | 0.264 | 0.015831135 |
| | Sorbate | | | | |
| | Sodium | 0.250 | 2.5 | 0.264 | 0.015831135 |
| | Citrate | | | | |
| | Magnasweet | 0.050 | 0.5 | 0.053 | 0.003166227 |
| Active | HMB | 7.94 | 79.4 | 8.380 | 0.502796834 |
| Ingredient | | | | | |
| Flavor | Mountain | 2.25 | 22.5 | 2.375 | 0.142480211 |
| System | Berry | | | | |
| | Mixed Berry | 2.25 | 22.5 | 2.375 | 0.142480211 |
| | Acid Flavor | 0.375 | 3.75 | 0.396 | 0.023746702 |
| | Reducer | | | | |
| | Totals | 100.00% | 1000 | 100.000% | 6 |
| | Material | | | pH | 4.51 |
| | Specs | | | Brix | 72 |

### Example 1b. Watermelon Flavor

| | | Slurry | | Final Product | |
|---|---|---|---|---|---|
| | | Formula | Formula | Formula | Formula |
| | Ingredient | (wt. %) | (g) | (wt. %) | (g) |
| Total Water | USP Water | 18 | 180.1 | 13.467 | 0.80802111 |
| Phase A | Water | — | 102.92715 | | |
| (Gelatin | Gelatin | 7.00 | 70 | 7.388 | 0.44327177 |
| Solution) | | | | | |
| Phase B | Sugar | 30.00 | 300 | 31.662 | 1.89973615 |
| (Sugar | Pectin | 1.00 | 10 | 1.055 | 0.06332454 |
| Solution) | USP Water | — | 77.17285 | | |
| | Tapioca Syrup | 22.00 | 220 | 23.219 | 1.39313984 |
| | Raspberry | 7.00 | 70 | 7.388 | 0.44327177 |
| | Concentrate | | | | |
| Solids (acids/ | Citric Acid | 1.00 | 10 | 1.055 | 0.06332454 |
| preservatives) | Malic Acid | 0.625 | 6.25 | 0.660 | 0.03957784 |
| | Potassium | 0.250 | 2.5 | 0.264 | 0.01583113 |
| | Sorbate | | | | |
| | Sodium Citrate | 0.250 | 2.5 | 0.264 | 0.01583113 |
| | Magnasweet | 0.050 | 0.5 | 0.05 | 0.00316623 |
| Active ingredient | HMB | 7.94 | 79.4 | 8.380 | 0.50279683 |
| Flavor system | Watermelon | 4.50 | 45 | 4.749 | 0.28496042 |
| | Flavor | | | | |
| | Acid Flavor | 0.375 | 3.75 | 0.396 | 0.0237467 |
| | Reducer | | | | |
| | Totals | 100.00 | 1,000 | 100.000 | 6 |
| | Material | | | pH | 4.51 |
| | Specs | | | Brix | 72 |

### Example 1c. Pineapple Flavor

| | | Slurry | | Final Product | |
|---|---|---|---|---|---|
| | | Formula | Formula | Formula | Formula |
| | Ingredient | (wt. %) | (g) | (wt. %) | (g) |
| Total Water | USP Water | 20 | 203.1 | 15.894 | 0.95366755 |
| Phase A | Water | — | 116.07165 | | |
| (Gelatin | Gelatin | 7.00 | 70 | 7.388 | 0.44327177 |
| Solution) | | | | | |
| Phase B | Sugar | 30.00 | 300 | 31.662 | 1.89973615 |
| (Sugar | Pectin | 1.00 | 10 | 1.055 | 0.06332454 |
| Solution) | USP Water | — | 87.02835 | | |
| | Tapioca Syrup | 22.00 | 220 | 23.219 | 1.39313984 |
| | Raspberry | 7.00 | 70 | 7.388 | 0.44327177 |
| | Concentrate | | | | |
| Solids (Acids/ | Citric Acid | 1.00 | 10 | 1.055 | 0.06332454 |
| Preservatives) | Malic Acid | 0.625 | 6.25 | 0.660 | 0.03957784 |
| | Potassium | 0.250 | 2.5 | 0.264 | 0.01583113 |
| | Sorbate | | | | |
| | Sodium Citrate | 0.250 | 2.5 | 0.264 | 0.01583113 |
| | Magnasweet | 0.050 | 0.5 | 0.053 | 0.00316623 |
| Active Ingredient | HMB | 7.94 | 79.4 | 8.380 | 0.50279683 |
| Flavor System | Pineapple | 2.20 | 22 | 2.322 | 0.13931398 |
| | Flavor | | | | |
| | Acid Flavor | 0.375 | 3.75 | 0.396 | 0.0237467 |
| | Reducer | | | | |
| | Totals | 100.00 | 1000 | 100.000 | 6 |
| | Material | | | pH | 4.51 |
| | Specs | | | Brix | 72 |

### Example 1d. Green Apple Flavor

| | | Slurry | | Final Product | |
|---|---|---|---|---|---|
| | | Formula | Formula | Formula | Formula |
| | Ingredient | (wt. %) | (g) | (wt. %) | (g) |
| Total Water | USP Water | 20 | 203.1 | 15.894 | 0.95366755 |
| Phase A | Water | — | 116.07165 | | |
| (Gelatin | Gelatin | 7.00 | 70 | 7.388 | 0.44327177 |
| Solution) | | | | | |
| Phase B | Sugar | 30.00 | 300 | 31.662 | 1.89973615 |
| (Sugar | Pectin | 1.00 | 10 | 1.055 | 0.06332454 |
| Solution) | USP Water | — | 87.02835 | | |
| | Tapioca Syrup | 22.00 | 220 | 23.219 | 1.39313984 |
| | Raspberry | 7.00 | 70 | 7.388 | 0.44327177 |
| | Concentrate | | | | |
| Solids (Acids/ | Citric Acid | 1.00 | 10 | 1.055 | 0.06332454 |
| Preservatives) | Malic Acid | 0.625 | 6.25 | 0.660 | 0.03957784 |
| | Potassium | 0.250 | 2.5 | 0.264 | 0.01583113 |
| | Sorbate | | | | |
| | Sodium Citrate | 0.250 | 2.5 | 0.264 | 0.01583113 |
| | Magnasweet | 0.050 | 0.5 | 0.053 | 0.00316623 |
| Active Ingredient | HMB | 7.94 | 79.4 | 8.380 | 0.50279683 |
| Flavor System | Green Apple | 2.20 | 22 | 2.322 | 0.13931398 |
| | Acid Flavor | 0.375 | 3.75 | 0.396 | 0.0237467 |
| | Reducer | | | | |
| | Totals | 100.00 | 1000 | 100.000 | 6 |
| | Material | | | pH | 4.51 |
| | Specs | | | Brix | 72 |

### Example 1E. Summary of Ingredients, Final Product

| Ingredient | Amount (wt. %) | Amount (g) |
|---|---|---|
| USP Water | 15 ± 5 | 0.90 ± 0.1 |
| Gelatin | 7.39 ± 1 | 0.44 ± 0.05 |
| Sugar | 31.66 ± 3 | 1.90 ± 0.2 |
| Pectin | 1.06 ± 0.1 | 0.06 ± 0.006 |
| Tapioca Syrup | 23.21 ± 2.5 | 1.40 ± 0.15 |
| Raspberry Concentrate | 7.39 ± 1 | 0.44 ± 0.05 |
| Citric Acid | 1.06 ± 1 | 0.06 ± 0.006 |
| Malic Acid | 0.66 ± 0.05 | 0.04 ± 0.005 |
| Potassium Sorbate | 0.26 ± 0.025 | 0.02 ± 0.002 |
| Sodium Citrate | 0.26 ± 0.025 | 0.02 ± 0.002 |
| Magnasweet MM100 | 0.05 ± 0.005 | 0.003 ± 0.0003 |
| HMB | 8.38 ± 1 | 0.50 ± 0.05 |
| Mountain Berry | 2.38 ± 2.38 | 0.14 ± 0.14 |
| Mixed Berry | 2.38 ± 2.38 | 0.14 ± 0.14 |
| Watermelon Flavor | 4.75 ± 4.75 | 0.28 ± 0.28 |
| Pineapple Flavor | 2.32 ± 2.32 | 0.14 ± 0.14 |
| Green Apple | 2.32 ± 2.32 | 0.14 ± 0.14 |
| Acid Flavor Reducer | 0.40 ± 0.05 | 0.02 ± 0.02 |
| TOTAL | 100 | 6 |

### Example 1f. Summary of Ingredients, Final Product

| Ingredient | Amount (wt. %) | Amount (g) |
|---|---|---|
| USP Water | 13.47-15.90 | 0.81-0.95 |
| Gelatin | 7.39 | 0.44 |
| Sugar | 31.66 | 1.90 |
| Pectin | 1.06 | 0.06 |
| Tapioca Syrup | 23.21-23.22 | 1.39-1.40 |
| Raspberry Concentrate | 7.39 | 0.44 |
| Citric Acid | 1.06 | 0.06 |
| Malic Acid | 0.66-0.67 | 0.04 |
| Potassium Sorbate | 0.26 | 0.02 |
| Sodium Citrate | 0.26 | 0.02 |
| Magnasweet MM100 | 0.05 | 0.003 |
| HMB | 8.38 | 0.50 |
| Mountain Berry | 0-2.38 | 0.14 |
| Mixed Berry | 0-2.38 | 0.14 |
| Watermelon Flavor | 0-4.75 | 0.28 |
| Pineapple Flavor | 0-2.32 | 0.14 |
| Green Apple | 0-2.32 | 0.14 |
| Acid Flavor Reducer | 0.40 | 0.02 |
| TOTAL | 100 | 6 |

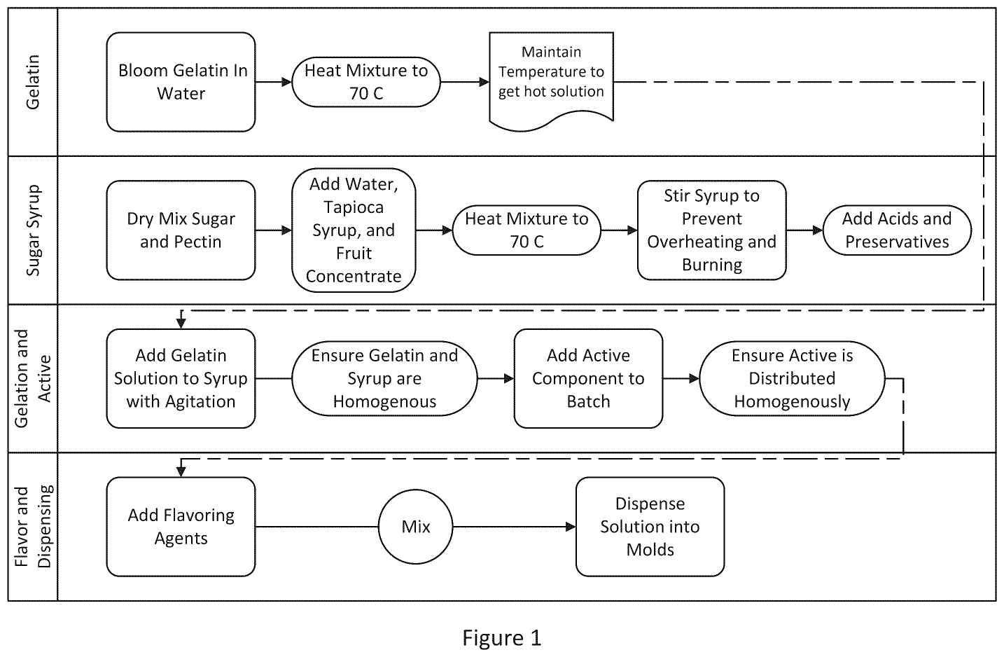

### Example 2. Chewable Gummy Containing β-Hydroxy β-Methylbutyric Acid (HMB), Method of Manufacturing as shown in FIG. 1

### Step 1

### Gelatin Preparation:

In an appropriately sized stainless steel induction pot dispense water. Place vessel on induction stove top (Induction Cooker BT-C35-D/P961LS) and set temperature to 70° C. As water is coming up to temperature add gelatin to pot and mix. Continue mixing as gelatin dissolves in water and solution comes up to temperature. Maintain solution at 60-70° C. until ready for next phase.

### Step 2

### Sugar Syrup Preparation:

In an appropriately sized stainless steel induction pot dispense sugar and pectin. Give sugar and pectin a dry mix to ensure even distribution of pectin. Add water, tapioca syrup, and fruit concentrate to induction pot. Place vessel on induction stove top (Induction Cooker BT-C35-D/P961LS) and set temperature to 70° C. Begin mixing to prevent burning of sugar. Continue mixing until mixture reaches 70° C. Once temperature is reached add acids and preservatives to pot and continue to mix until homogenous.

### Step 3

### Gelatin and Active Addition:

Once Sugar Syrup solution is homogenous add heated gelatin solution to vessel with agitation. Continue mixing gelatin into sugar syrup. Once gelatin and syrup are homogenous add HMB free acid to induction pot. Check temperature and ensure temp is within 60-70° C. Mix HMB free acid into pot until homogenous.

### Step 4

### Addition of Flavor

Once all other ingredients are in the solution flavors may be added. Add flavoring components into solution. Mix flavor into solution for five minutes. Once flavor has been mixed test pH and Brix of solution. Dispense solution into molds and allow to cure. Cure at 20-22° C. at relative humidity (RH) at 40%. Gummies will cure in 6 hours but can continue to cure for 24 hours.

1. A chewable gummy comprising:
β-hydroxy β-methylbutyric acid (HMB);
gelling agent comprising at least one of gelatin and pectin;
sweetening agent comprising at least one of sugar, tapioca syrup, and magnasweet MM100;
flavoring agent comprising at least one of raspberry flavor, green apple flavor, mountain berry flavor, mixed berry flavor, pineapple flavor, watermelon flavor, citric acid, malic acid, and acid flavor reducer;
preservative comprising at least one of potassium sorbate and sodium citrate, and
solvent comprising water;
wherein,
the chewable gummy is configured as a unit dose having a mass of 6±1 g;
the pH is 4.51±0.5;
the Brix is 72±7; and
the water content is 12-17 wt. %.

2. The chewable gummy of claim 1, wherein the β-hydroxy β-methylbutyric acid (HMB) is in the free acid form, β-hydroxy β-methylbutyric acid (HMB-FA).

3. The chewable gummy of claim 1, wherein the β-hydroxy β-methylbutyric acid (HMB) is in the monohydrated calcium salt of the conjugate base form, calcium β-hydroxy β-methylbutyrate monohydrate (HMB-Ca, CaHMB).

4. The chewable gummy of claim 1, wherein the β-hydroxy β-methylbutyric acid (HMB) is present in 8.3±1 wt. %.

5. The chewable gummy of claim 1, wherein the β-hydroxy β-methylbutyric acid (HMB) is present in 0.5±0.05 g.

6. The chewable gummy of claim 1, wherein the gelling agent comprises gelatin and pectin.

7. The chewable gummy of claim 1, wherein the gelling agent comprises gelatin and pectin, present in a combined amount of 0.5±0.05 g.

8. The chewable gummy of claim 1, wherein the gelling agent comprises gelatin and pectin, present in a combined amount of 8.4±0.8 wt. %.

9. The chewable gummy of claim 1, wherein the sweetening agent comprises sugar, tapioca syrup, and magnasweet MM100.

10. The chewable gummy of claim 1, wherein the sweetening agent comprises sugar, tapioca syrup, and magnasweet MM100, present in a combined amount of 3.3±0.5 g.

11. The chewable gummy of claim 1, wherein the sweetening agent comprises sugar, tapioca syrup, and magnasweet MM100, present in a combined amount of 54.9±5 wt. %.

12. The chewable gummy of claim 1, wherein the flavoring agent comprises citric acid, malic acid, and acid flavor reducer, present in a combined amount of 0.13±0.01 g.

13. The chewable gummy of claim 1, wherein the flavoring agent comprises citric acid, malic acid, and acid flavor reducer, present in a combined amount of 2.1±0.2 wt. %.

14. The chewable gummy of claim 1, wherein the flavoring agent comprises raspberry flavor, mountain berry flavor, and mixed berry flavor.

15. The chewable gummy of claim 1, wherein the flavoring agent comprises raspberry flavor and watermelon flavor.

16. The chewable gummy of claim 1, wherein the flavoring agent comprises raspberry flavor and pineapple flavor.

17. The chewable gummy of claim 1, wherein the flavoring agent comprises raspberry flavor and green apple flavor.

18. The chewable gummy of claim 1, wherein the water content is 13-16 wt. %.

19. The chewable gummy of claim 1, wherein the pH is 4.51±0.25.

20. The chewable gummy of claim 1, wherein the Brix is 72±5.

21. The chewable gummy of claim 1, comprising:

| Ingredient | Amount (wt. %) | Amount (g) |
|---|---|---|
| USP Water | 13.47 ± 1.5 | 0.81 ± 0.1 |
| Gelatin | 7.39 ± 0.8 | 0.44 ± 0.05 |
| Sugar | 31.66 ± 4 | 1.90 ± 0.2 |
| Pectin | 1.06 ± 0.2 | 0.06 ± 0.01 |
| Tapioca Syrup | 23.21 ± 3 | 1.39 ± 0.2 |
| Raspberry Concentrate | 7.39 ± 1 | 0.44 ± 0.05 |
| Citric Acid | 1.06 ± 0.2 | 0.06 ± 0.01 |
| Malic Acid | 0.66 ± 0.1 | 0.04 ± 0.01 |
| Potassium Sorbate | 0.26 ± 0.05 | 0.02 ± 0.005 |
| Sodium Citrate | 0.26 ± 0.05 | 0.02 ± 0.005 |
| Magnasweet MM100 | 0.05 ± 0.01 | 0.003 ± 0.001 |
| HMB | 8.38 ± 1 | 0.50 ± 0.05 |
| Mountain Berry | 2.38 ± 0.3 | 0.14 ± 0.02 |
| Mixed Berry | 2.38 ± 0.3 | 0.14 ± 0.02 |
| Acid Flavor Reducer | 0.40 ± 0.05 | 0.02 ± 0.003 |
| Total | 100 | 6 ± 0.75 |

22. The chewable gummy of claim 1, comprising:

| Ingredient | Amount (wt. %) | Amount (g) |
|---|---|---|
| USP Water | 13.47 | 0.81 |
| Gelatin | 7.39 | 0.44 |
| Sugar | 31.66 | 1.90 |
| Pectin | 1.06 | 0.06 |
| Tapioca Syrup | 23.21 | 1.39 |
| Raspberry Concentrate | 7.39 | 0.44 |
| Citric Acid | 1.06 | 0.06 |
| Malic Acid | 0.66 | 0.04 |
| Potassium Sorbate | 0.26 | 0.02 |
| Sodium Citrate | 0.26 | 0.02 |
| Magnasweet MM100 | 0.05 | 0.003 |
| HMB | 8.38 | 0.50 |
| Mountain Berry | 2.38 | 0.14 |
| Mixed Berry | 2.38 | 0.14 |
| Acid Flavor Reducer | 0.40 | 0.02 |
| Total | 100 | 6 |

23. The chewable gummy of claim 1, comprising:

| Ingredient | Amount (wt. %) | Amount (g) |
|---|---|---|
| USP Water | 13.47 ± 1.5 | 0.81 ± 0.1 |
| Gelatin | 7.39 ± 1 | 0.44 ± 0.1 |
| Sugar | 31.66 ± 4 | 1.90 ± 0.3 |
| Pectin | 1.06 ± 0.2 | 0.06 ± 0.01 |
| Tapioca Syrup | 23.21 ± 3 | 1.40 ± 0.3 |
| Raspberry Concentrate | 7.39 ± 1 | 0.44 ± 0.05 |
| Citric Acid | 1.06 ± 0.2 | 0.06 ± 0.01 |
| Malic Acid | 0.67 ± 0.1 | 0.04 ± 0.01 |
| Potassium Sorbate | 0.26 ± 0.05 | 0.02 ± 0.005 |
| Sodium Citrate | 0.26 ± 0.05 | 0.02 ± 0.005 |
| Magnasweet MM100 | 0.05 ± 0.01 | 0.003 ± 0.001 |
| HMB | 8.38 ± 1 | 0.50 ± 0.1 |
| Watermelon Flavor | 4.75 ± 0.5 | 0.28 ± 0.05 |
| Acid Flavor Reducer | 0.40 ± 0.05 | 0.02 ± 0.005 |
| Total | 100 | 6 ± 0.75 |

24. The chewable gummy of claim 1, comprising:

| Ingredient | Amount (wt. %) | Amount (g) |
|---|---|---|
| USP Water | 13.47 | 0.81 |
| Gelatin | 7.39 | 0.44 |
| Sugar | 31.66 | 1.90 |
| Pectin | 1.06 | 0.06 |
| Tapioca Syrup | 23.21 | 1.40 |
| Raspberry Concentrate | 7.39 | 0.44 |
| Citric Acid | 1.06 | 0.06 |
| Malic Acid | 0.67 | 0.04 |
| Potassium Sorbate | 0.26 | 0.02 |
| Sodium Citrate | 0.26 | 0.02 |
| Magnasweet MM100 | 0.05 | 0.003 |
| HMB | 8.38 | 0.50 |
| Watermelon Flavor | 4.75 | 0.28 |
| Acid Flavor Reducer | 0.40 | 0.02 |
| Total | 100 | 6 |

25. The chewable gummy of claim 1, comprising:

| Ingredient | Amount (wt. %) | Amount (g) |
|---|---|---|
| USP Water | 15.90 ± 1.5 | 0.95 ± 0.1 |
| Gelatin | 7.39 ± 1 | 0.44 ± 0.1 |
| Sugar | 31.66 ± 3 | 1.90 ± 0.2 |
| Pectin | 1.06 ± 0.1 | 0.06 ± 0.01 |
| Tapioca Syrup | 23.22 ± 3 | 1.40 ± 0.2 |
| Raspberry Concentrate | 7.39 ± 1 | 0.44 ± 0.05 |
| Citric Acid | 1.06 ± 0.1 | 0.06 ± 0.01 |
| Malic Acid | 0.66 ± 0.1 | 0.04 ± 0.01 |
| Potassium Sorbate | 0.26 ± 0.03 | 0.02 ± 0.005 |
| Sodium Citrate | 0.26 ± 0.03 | 0.02 ± 0.005 |
| Magnasweet MM100 | 0.05 ± 0.01 | 0.003 ± 0.001 |
| HMB | 8.38 ± 1 | 0.50 ± 0.1 |
| Pineapple Flavor | 2.32 ± 0.3 | 0.14 ± 0.02 |
| Acid Flavor Reducer | 0.40 ± 0.1 | 0.02 ± 0.005 |
| Total | 100 | 6 ± 0.75 |

26. The chewable gummy of claim 1, comprising:

| Ingredient | Amount (wt. %) | Amount (g) |
|---|---|---|
| USP Water | 15.90 | 0.95 |
| Gelatin | 7.39 | 0.44 |
| Sugar | 31.66 | 1.90 |
| Pectin | 1.06 | 0.06 |
| Tapioca Syrup | 23.22 | 1.40 |
| Raspberry Concentrate | 7.39 | 0.44 |
| Citric Acid | 1.06 | 0.06 |
| Malic Acid | 0.66 | 0.04 |
| Potassium Sorbate | 0.26 | 0.02 |
| Sodium Citrate | 0.26 | 0.02 |
| Magnasweet MM100 | 0.05 | 0.003 |
| HMB | 8.38 | 0.50 |
| Pineapple Flavor | 2.32 | 0.14 |
| Acid Flavor Reducer | 0.40 | 0.02 |
| Total | 100 | 6 |

27. The chewable gummy of claim 1, comprising:

| Ingredient | Amount (wt. %) | Amount (g) |
|---|---|---|
| USP Water | 15.90 ± 1.5 | 0.95 ± 0.1 |
| Gelatin | 7.39 ± 1 | 0.44 ± 0.05 |
| Sugar | 31.66 ± 3 | 1.90 ± 0.2 |
| Pectin | 1.06 ± 0.1 | 0.06 ± 0.01 |
| Tapioca Syrup | 23.22 ± 3 | 1.40 ± 0.2 |
| Raspberry Concentrate | 7.39 ± 1 | 0.44 ± 0.1 |
| Citric Acid | 1.06 ± 0.1 | 0.06 ± 0.01 |
| Malic Acid | 0.66 ± 0.1 | 0.04 ± 0.01 |
| Potassium Sorbate | 0.26 ± 0.05 | 0.02 ± 0.005 |
| Sodium Citrate | 0.26 ± 0.05 | 0.02 ± 0.005 |
| Magnasweet MM100 | 0.05 ± 0.01 | 0.003 ± 0.001 |
| HMB | 8.38 ± 1 | 0.50 ± 0.05 |
| Green Apple | 2.32 ± 0.3 | 0.14 ± 0.02 |
| Acid Flavor Reducer | 0.40 ± 0.1 | 0.02 ± 0.002 |
| Total | 100 | 6 ± 0.75 |

28. The chewable gummy of claim 1, comprising:

| Ingredient | Amount (wt. %) | Amount (g) |
|---|---|---|
| USP Water | 15.90 | 0.95 |
| Gelatin | 7.39 | 0.44 |
| Sugar | 31.66 | 1.90 |
| Pectin | 1.06 | 0.06 |
| Tapioca Syrup | 23.22 | 1.40 |
| Raspberry Concentrate | 7.39 | 0.44 |
| Citric Acid | 1.06 | 0.06 |
| Malic Acid | 0.66 | 0.04 |
| Potassium Sorbate | 0.26 | 0.02 |
| Sodium Citrate | 0.26 | 0.02 |
| Magnasweet MM100 | 0.05 | 0.003 |
| HMB | 8.38 | 0.50 |
| Green Apple | 2.32 | 0.14 |
| Acid Flavor Reducer | 0.40 | 0.02 |
| Total | 100 | 6 |

29. The chewable gummy of claim 1, configured as a dietary supplement.

30. The chewable gummy of claim 1, configured as a medical food.

31. A method comprising orally administering the chewable gummy of claim 1 to a human subject.

32. The method of claim 31
which promotes wound healing to the human subject following surgery or injury;
which provides nutritional support to the human subject;
which provides nutritional support to the human subject with muscle wasting due to cancer or HIV/AIDS;
which (a) increases exercise-induced gains in muscle size, muscle strength, and/or lean body mass, (b) reduces skeletal muscle damage from exercise, (c) improves aerobic exercise performance, and/or (d) expedites recovery from exercise;
which preserves and/or increases lean body mass and muscle strength in the human subject experiencing age-related muscle loss;
which preserves lean body mass in muscle wasting conditions;
which preserves lean body mass in human subjects afflicted with sarcopenia, or is at risk thereof;
which prevents or mitigates muscle atrophy during bed rest;
which reduces the loss of muscle mass, muscle function, and muscle strength that occurs in hypercatabolic disease states, such as cancer cachexia;
which is an adjunct therapy in conjunction with resistance exercise;
which is an adjunct therapy in conjunction with resistance exercise and consumption of a high-protein diet;
which (a) increases exercise-induced gains in muscle size, muscle strength and power, (b) increases lean body mass, (c) reduces exercise-induced skeletal muscle damage, and/or (d) expedites recovery from high-intensity exercise;
which improves aerobic exercise performance and increases gains in aerobic fitness when combined with high-intensity interval training; and/or
which reduces skeletal muscle damage from prolonged or high-intensity exercise.

33. The method of claim 31, in which 3-6 grams of HMB per day are orally administered to the human subject.

34. The method of claim 31, wherein 1-3 chewable gummies are orally administered to the human subject 0.25-2.5 hours prior to exercise.

35. The method of claim 31, wherein 1-3 chewable gummies are orally administered to the human subject 0.5-2 hours prior to exercise.

36. The method of claim 31, in which a total of 1-15 chewable gummies are orally administered to the human subject throughout the day.
